(19) United States
(12) Patent Application Publication
(10) Pub. No.: US 2023/0298762 A1
(43) Pub. Date: Sep. 21, 2023

(54) A SYSTEM AND METHOD FOR SELF-ASSESSMENT OF DENTAL PROBLEMS

(71) Applicant: Manoj RAJAN, Chennai (IN)

(72) Inventor: Manoj RAJAN, Chennai (IN)

(21) Appl. No.: 18/202,718

(22) Filed: May 26, 2023

Related U.S. Application Data
(63) Continuation of application No. PCT/IB2021/061102, filed on Nov. 30, 2021.

(30) Foreign Application Priority Data
Nov. 30, 2020 (IN) 202041052169

(51) Int. Cl. G16H 50/30; G16H 10/60; A61B 1/24

(52) U.S. Cl. CPC G16H 50/30; G16H 10/60; A61B 1/24

(57) ABSTRACT

There is provided a system and method for self-assessment of dental problems that can help in monitoring and improving dental hygiene. The system comprises a camera adapted to capture a plurality of images and/or videos of teeth of a user from multiple angles; a user interface to receive one or more inputs related to a medical condition and habits of the user affecting the teeth; and a processing module connected with the camera and the user interface. The proposed system and method provide a handy solution to the people to keep a check on their dental health themselves.

### CROSS-REFERENCE TO RELATED APPLICATIONS

This is a continuation application under 35 U.S.C. § 120 of International Application PCT/IB2021/061102, filed Nov. 30, 2021, and claims priority to IN Application 202041052169, filed Nov. 30, 2020, the contents of each being incorporated by reference herein.

### FIELD OF THE INVENTION

Embodiments of the present invention generally relate to dental health and hygiene. More particularly, the present invention relates to a system and method for self-assessment of dental problems that can help in monitoring and improving dental hygiene.

### BACKGROUND OF THE INVENTION

The subject matter discussed in the background section should not be assumed to be prior art merely as a result of it being mentioned in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

Dental health problems have been increasing throughout the globe and reasons are numerous. The incidence of dental cavities and dental tartar are increasing in the world in spite of better quality of care and dental insurance. But it is globally accepted notion that dental health can be secured by brushing teeth regularly and undergoing regular dental check-ups. However, it is a well-known fact that not everybody follows the above-mentioned practice honestly and regularly. It is a tendency of humans to pay attention to health (especially dental health which is often ignored) only when the problem arises or deteriorates. Regular check-up to prevent any possible problems is avoided most of the time. Also, the examination of the teeth has to be carried out by a dentist with expertise in dentistry, but the cost of dental examinations, which are spent at regular intervals, can be an economic burden for the general public. Additionally, it is not always possible for a person to visit the dentist all the time for a regular check-up or for every small pain.

Neither is dental health easy to understand for general public, who have a habit of treating physical health problems using home remedies or commonly prescribed medicines. Furthermore, during the unprecedented times like the ongoing pandemic, where the whole world has come to a halt, visiting a dentist is not only dangerous for the patient but is also not safe for the dentist to attend the patients. At present, even in this digital age, there are no options for a person to even assess their dental health themselves. There are no solutions available that can monitor and tell identify whether the dental health of person is sound, or has small issues like dental plaque, cavities etc. So, they have no option but to visit a dentist even for a general check-up. If the visit is not feasible or dentist is not available, they are on their own. Studies have shown that cavities and tartar start small but progress to become large and cause damage to teeth quite quickly.

Moreover, there can be another scenario for dental health insurance companies, that often require prior check-up of the teeth before issuing an insurance policy etc. In the ongoing pandemic situation, it is not feasible for a person to go for medical (dental) check-up just to complete the formality for buying an insurance. So, even in that scenario there are no solution available that can enable a person to self-assess his/her dental health and submit dental assessment report to such insurance companies, to complete the formalities. This could also be used by expert dentists to identify pre-existing problems before the insurance. Lack of such self-assessment solutions, later on cause dental problems to the people and that in turn cost the insurance companies huge amount money because of the insurance claims.

Therefore, there is a need in the art for a system and method for self-assessment of dental problems that can help in monitoring and improving dental hygiene. The solution should be cost-effective and feasible even in the unprecedented times of pandemic.

### OBJECT OF THE INVENTION

An object of the invention is to provide a system and method for self-assessment of dental problems that can help in monitoring and improving dental hygiene.

Another objective of the invention is to utilize image processing technologies, Artificial Intelligence and machine learning to assess and improve dental hygiene.

Yet another objective of the invention is to provide a handy solution to the people to keep a check on their dental health themselves.

Yet another object of the present invention is to provide dental assessment results of self-assessment of dental health by the insurers to remotely completely the insurance formalities and to dynamically underwrite insurance pricing.

Yet another object of the present invention is to provide a cost-effective solution for the dental professionals that can be used access dental patients as a pre-check-up and routine checks, even in the unprecedented times like the ongoing pandemic.

### SUMMARY OF THE INVENTION

Embodiments of the present invention provide a system and method for self-assessment of dental problems that can help in monitoring and improving dental hygiene. The proposed system and method provides a handy solution to the people to keep a check on their dental health themselves. The present system and method will be cross platform supported and can run on any operating system, but not limited to, Android, iOS, Windows etc. However, it should be noted that the present invention should not be confused with a tool for diagnosis or treatment of dental problems

According to a first aspect of the present invention, there is provided a system for self-assessment of dental problems. The system comprises a camera adapted to capture a plurality of images and/or videos of teeth of a user from multiple angles; a user interface to receive one or more inputs related to a medical condition and habits of the user affecting the teeth; and a processing module connected with the camera and the user interface. Herein, the processing module is configured to receive the plurality of images and/or videos of the teeth of the user from multiple angles; receive one or more inputs related to existing health conditions and habits of the user possibly affecting the teeth; process the plurality of images and/or videos to identify dental condition & possible problems and marking dental risk area, if any, on the received the plurality of images and/or videos; correlate the identified dental condition & problems with the existing medical condition and habits of the user and accordingly generate assessment details along with a risk assessment score; and provide the assessment details and the risk assessment score along with marked images and/or videos showing the dental risk areas on the user interface.

In accordance with an embodiment of the present invention, the existing health conditions possibly affecting the teeth, include high blood pressure, heart disease, diabetes, long-term kidney disease, lung disease, obesity, osteoporosis, rheumatoid arthritis, HIV and anemia.

In accordance with an embodiment of the present invention, the habits of the user possibly affecting the teeth are selected from one or more of brushing habits, flossing habits, smoking, alcohol consumption, tobacco consumption, excessive coffee consumption, binge eating, inadequate hydration, grinding the teeth, misuse of tooth picks and nail biting.

In accordance with an embodiment of the present invention, the processing module is adapted to identify the dental condition and possible problems in permanent teeth, deciduous teeth, and teeth with braces or any form of aligners and teeth with dental implants including all fixed and removable prosthesis.

In accordance with an embodiment of the present invention, the dental condition and problems include dental cavities, dental plaque & tartar, missing teeth, gingivitis, bleeding gums, gum recession, gaps in the teeth, crooked teeth, teeth positioning, chipped or worn-out teeth, teeth discoloration and jaw alignment.

In accordance with an embodiment of the present invention, the assessment details include details of the identified dental condition and problems; correlation with the existing medical condition and habits; possible future problems in teeth; corrective changes in lifestyle and habits to improve oral hygiene; and if required, advice to seek consult a dentist.

In accordance with an embodiment of the present invention, the risk assessment score is generated by assigning a separate risk score to each identified dental condition & problem, and then generating an overall dental risk score also taking into consideration the existing medical condition and habits, thereby enabling the user to identify high, medium and low risk conditions.

In accordance with an embodiment of the present invention, the processing module is configured to enable the user to track and monitor the improvement of the respective oral hygiene by keeping results of previous assessments and providing a comparative analysis of the present assessment results with the previous assessment results.

In accordance with an embodiment of the present invention, the camera is selected from camera provided in a mobile computing device such as a smartphone, tablet or laptop; and a dedicated camera device such as DSLR, digital cameras, mirror less cameras and webcams.

In accordance with an embodiment of the present invention, the processing module is also configured to utilize sensory inputs from one or more of trans illumination lights, ultrasound or Infrared sensors, accelerometers and gyro sensors available in the smartphones or dedicated external sensors connected with processing module.

According to a second aspect of the present invention, there is provided a method for self-assessment of dental problems. The method comprises receiving a plurality of images and/or videos of the teeth of the user from multiple angles captured from a camera; receiving, from a user interface, one or more inputs related to existing health conditions and habits of the user possibly affecting the teeth; processing the plurality of images and/or videos to identify dental condition and possible problems and marking dental risk area, if any, on the received the plurality of images and/or videos; correlating the identified dental condition and problems with the existing medical condition and habits of the user and accordingly generating assessment details along with a risk assessment score; and providing the assessment details and the risk assessment score along with marked images and/or videos showing the dental risk areas on the user interface.

### BRIEF DESCRIPTION OF THE DRAWINGS

So that the manner in which the above recited features of the present invention can be understood in detail, a more particular to the description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, the invention may admit to other equally effective embodiments.

These and other features, benefits and advantages of the present invention will become apparent by reference to the following text figure, with like reference numbers referring to like structures across the views, wherein:

FIG. 1 illustrates a system for self-assessment of dental problems, in accordance with an embodiment of the present invention;

FIG. 2 illustrates a method for self-assessment of dental problems, in accordance with an embodiment of the present invention;

FIG. 3A-3B illustrates information flow diagrams of an exemplary implementation of system and method of FIGS. 1 and 2, in accordance with an embodiment of the present invention; and

FIG. 4A-4G illustrates screenshots of the exemplary implementation in a mobile computing device of a user, in accordance with an embodiment of the present invention.

### DETAILED DESCRIPTION OF THE DRAWINGS

The present invention is described hereinafter by various embodiments. This invention may, however, be embodied in many different forms and should not be interpreted as limited to the embodiment set forth herein. Rather, the embodiment is provided so that this disclosure will be thorough and complete and will fully convey the scope of the invention to those skilled in the art.

While the present invention is described herein by way of example using embodiments and illustrative drawings, those skilled in the art will recognize that the invention is not limited to the embodiments of drawing or drawings described and are not intended to represent the scale of the various components. Further, some components that may form a part of the invention may not be illustrated in certain figures, for ease of illustration, and such omissions do not limit the embodiments outlined in any way. It should be understood that the drawings and detailed description thereto are not intended to limit the invention to the particular form disclosed, but on the contrary, the invention is to cover all modifications, equivalents, and alternatives falling within the scope of the present invention as defined by the appended claim. As used throughout this description, the word “may” is used in a permissive sense (i.e., meaning having the potential to), rather than the mandatory sense, (i.e., meaning must). Further, the words “a” or “an” mean “at least one” and the word “plurality” means “one or more” unless otherwise mentioned. Furthermore, the terminology and phraseology used herein is solely used for descriptive purposes and should not be construed as limiting in scope. Language such as “including,” “comprising,” “having,” “containing,” or “involving,” and variations thereof, is intended to be broad and encompass the subject matter listed thereafter, equivalents, and additional subject matter not recited, and is not intended to exclude other additives, components, integers or steps. Likewise, the term “comprising” is considered synonymous with the terms “including” or “containing” for applicable legal purposes.

Embodiments of the present invention provide a system and method for self-assessment of dental problems that can help in monitoring and improving dental hygiene. The proposed system and method provide a handy solution to the people to keep a check on their dental health themselves. The present system and method will be cross platform supported and can run on any operating system, but not limited to, android, iOS, Windows etc. However, it should be noted that the present invention should not be confused with a tool for diagnosis or treatment of dental problems. It will be appreciated by a skilled addressee that the present invention is based on image processing and Machine Learning, so the dental health check is not restricted to humans only. In some embodiments, where the system is trained accordingly, the present invention may also easily be implemented for checking dental health of animals.

The present invention will be described hereinafter by various embodiments with reference to the accompanying drawings, wherein reference numerals used in the accompanying drawing correspond to the like elements throughout the description.

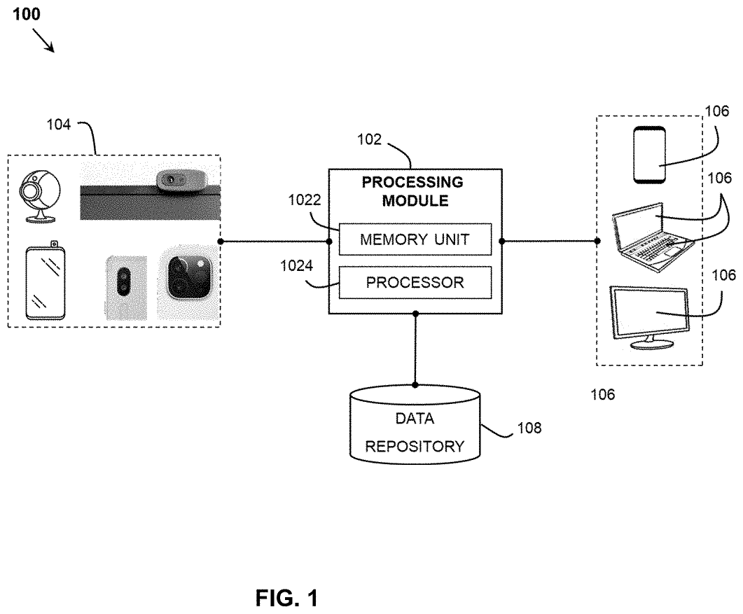

FIG. 1 illustrates a system (100) for self-assessment of dental problems, in accordance with an embodiment of the present invention. Herein, the system (100) is capable of identifying the dental condition and possible problems in permanent teeth, deciduous teeth, teeth with braces or any form of aligners and teeth with dental implants including all fixed and removable prosthesis. Also, the dental problems that may be identified by the present invention include, but not limited to, dental cavities, dental plaque & tartar, missing teeth, gingivitis, bleeding gums, gum recession, gaps in the teeth, crooked teeth, teeth positioning, chipped or worn-out teeth, teeth discoloration and jaw alignment.

The system (100) comprises, but not limited to, a camera (104) and a user interface (106) connected with processing module (102). The camera (104) may be, but not limited to, a camera (104) provided in a mobile computing device such as a smartphone, tablet, laptop etc. or may be a dedicated camera device (104) such as DSLR camera, digital camera, mirrorless camera, webcams etc. The camera (104) is adapted to capture a plurality of images and/or videos of teeth of a user from multiple angles. Further, the user interface (106) may be understood as an input-output means for the system (100). The user interface (106) enables the user to interact with the system (100), receive input and display output. In that sense, the user interface (106) may be, but not limited to, a touch-input screen of a smartphone/tablet, or a combination of screen, keyboard and trackpad/mouse of a laptop or a desktop PC.

Additionally, the processing module (102) is envisaged to include computing capabilities such as a memory unit (1022) configured to store machine readable instructions. The machine-readable instructions may be loaded into the memory unit (1022) from a non-transitory machine-readable medium, such as, but not limited to, CD-ROMs, DVD-ROMs and Flash Drives. Alternately, the machine-readable instructions may be loaded in a form of a computer software program into the memory unit (1022). The memory unit (1022) in that manner may be selected from a group comprising EPROM, EEPROM and Flash memory. Furthermore, the processing module (102) includes a processor (1024) operably connected with the memory unit (1022). Further, the processing module (102) may include a processor or plurality of high-speed computing processors with multiple cores operably connected with the memory unit (1022). In various embodiments, the processor may be a microprocessor selected from one of, but not limited to, an ARM based or Intel based processor or in the form of field-programmable gate array (FPGA), a general-purpose processor (GPP) and an application specific integrated circuit (ASIC).

Moreover, the processing module (102) may implement artificial intelligence, machine learning and deep learning-based technologies for, but not limited to, data analysis, collating data & presentation of data in real-time.

In accordance with an embodiment of the present invention, a communication network may also be used in the system (100) for connecting with a remote analytic system or for connecting the components of the system (100) to implement it as a distributed system. The communication network can be a short-range communication network and/or a long-range communication network, wire or wireless communication network. The communication interface includes, but not limited to, a serial communication interface, a parallel communication interface or a combination thereof. The communication network may be implemented using a number of protocols, such as but not limited to, TCP/IP, 3GPP, 3GPP2, LTE, IEEE 802.x etc. The communication network may be a wireless communication network selected from one of, but not limited to, internet network providing maximum coverage. For this purpose, an internet module connected through a wire or wireless module with the external plurality of internet access point or Internet Service Provider (ISP) may be provided in the system (100). The internet module is configured to establish the communication network (internet) access to the system (100). In one embodiment, the internet module enables a communication of the system (100) to external cloud applications, cloud storage and/or cloud processing servers.

In one embodiment, there may be a data repository (108) in the system (100). The data repository (108) may be a local storage (such as SSD, eMMC, Flash, SD card, etc) or a cloud-based storage. In any manner, the data repository (108) is envisaged to be capable of providing the data to the processing module (102), when the data is queried appropriately using applicable security and other data transfer protocols. The data repository (108) is configured to store the data including images/videos related to various dental conditions and indications thereof, such as teeth having cavities, plaque and other problems, individually or in combination, from multiple angles. In one embodiment, the data repository (108) is envisaged to store one or more trained machine learning models generated with the help of thousands of images of teeth from multiple angles indicative of various dental conditions, symptoms and results thereof, using machine learning and deep learning techniques. The data repository (108) may also be configured to store the assessment details of respective users and can provide the same when queried appropriately using applicable security and other data transfer protocols, to enable the users to track, compare and monitor their dental hygiene with respect to old assessment details.

It will be appreciated by a skilled addressee that the present invention is based on image processing and Machine Learning, so the dental health check is not restricted to humans only. In some embodiments, one or more trained machine learning models may be generated using dental images of animals, thereby enabling the present invention to be implemented for checking dental health of animals by their owners, without requiring a veterinary.

In one aspect, the system (100) may be implemented in an embedded system (100) having the camera (104), the user interface (106), the data repository (108) and the processing module (102) connected with the communication network, in a single device such as smartphone, tablet or laptop. In another aspect, the system (100) may be a distributed system (100) with the camera (104) being externally disposed and connected with the processing module (102), the user interface (106) & the storage module in a separate computing device. Desktop PC connected with a webcam, may be an example of one such system (100). In yet another aspect, the processing may be done on the remote analytics (cloud processing) server via the communication network using the images/videos from the smartphone, laptop, tablet etc. having the camera (104). In yet another embodiment of a remote analytics server based system (100), the processing module (102) itself may be a part of the cloud processing server and is connected to the user device (smartphone, laptop, tablet, PC etc.) via the communication network. A person skilled in the art would appreciate that the system (100) may be implemented in a plurality of ways, without departing from the scope of the present invention.

In accordance with an embodiment of the present invention, apart from images, the system (100) may also utilize sensory inputs from one or more of transillumination lights, ultrasound or InfraRed sensors, accelerometers and gyro sensors available in the smartphones, or dedicated external sensors connected with processing module (102).

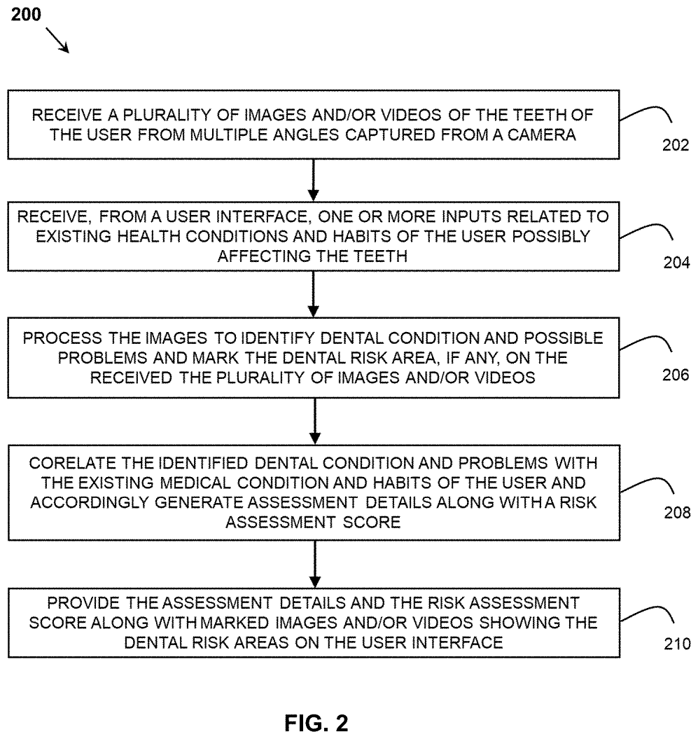

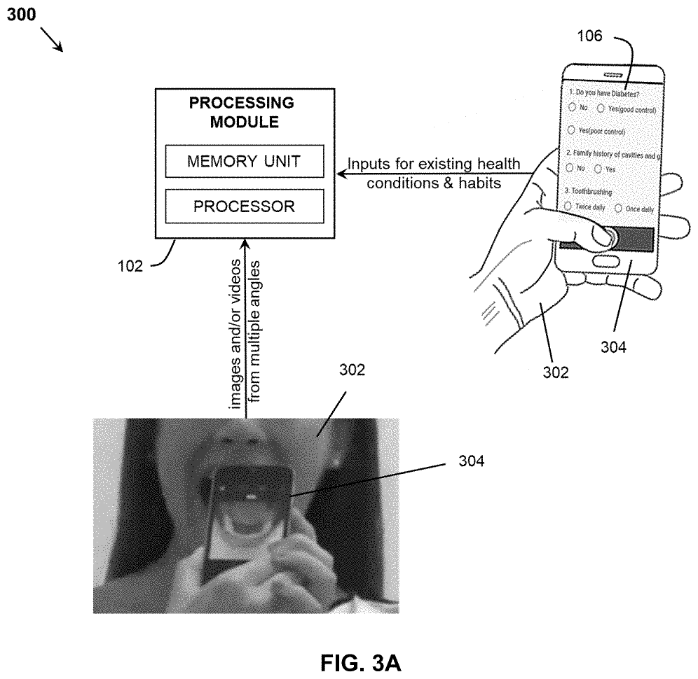

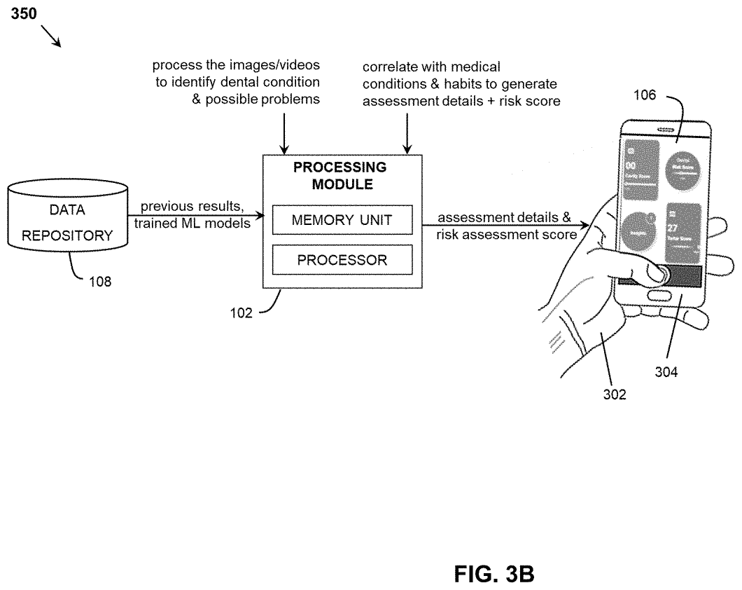

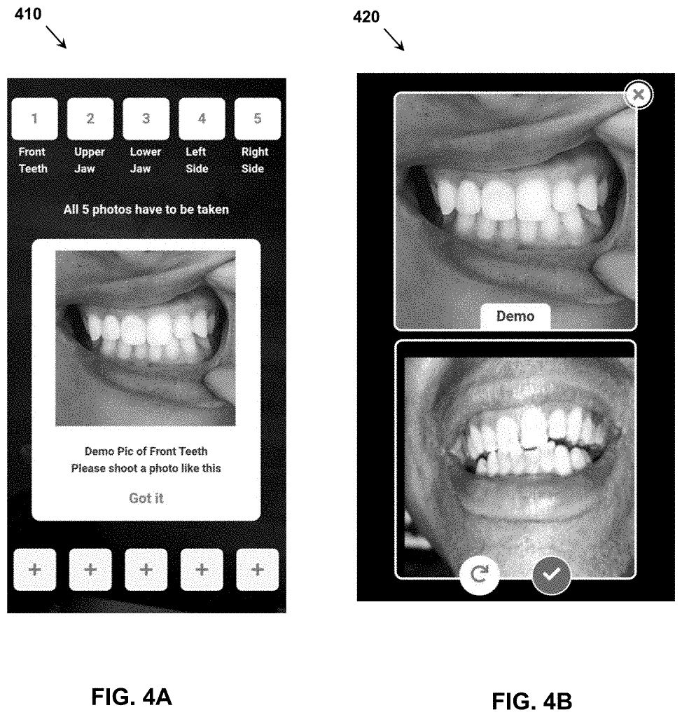

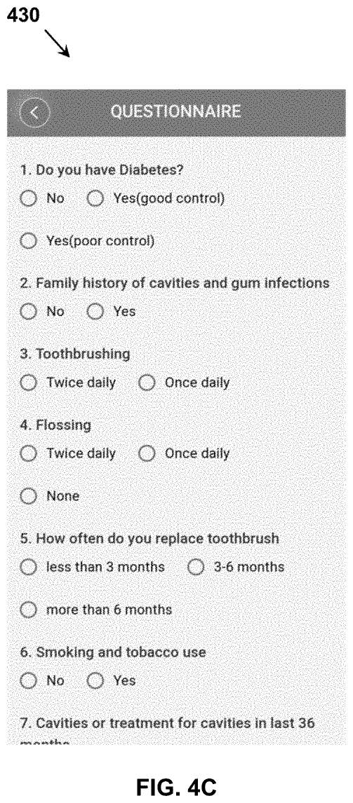

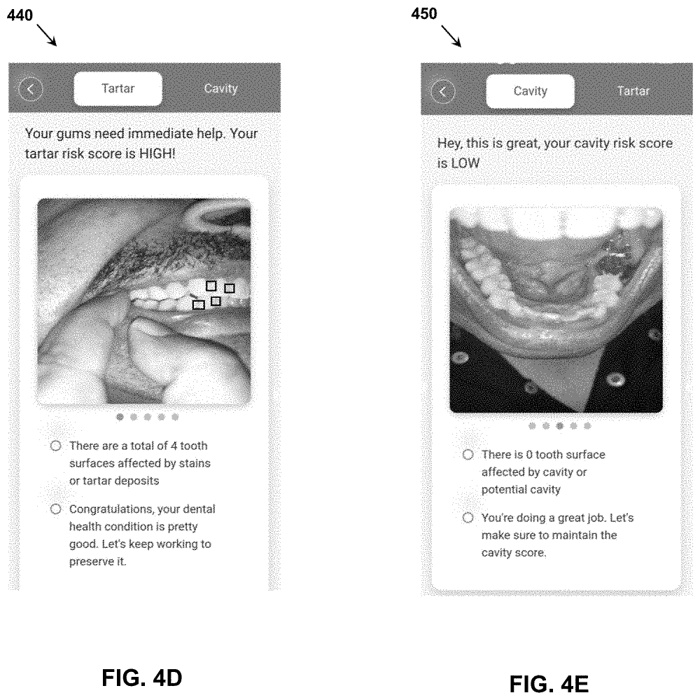

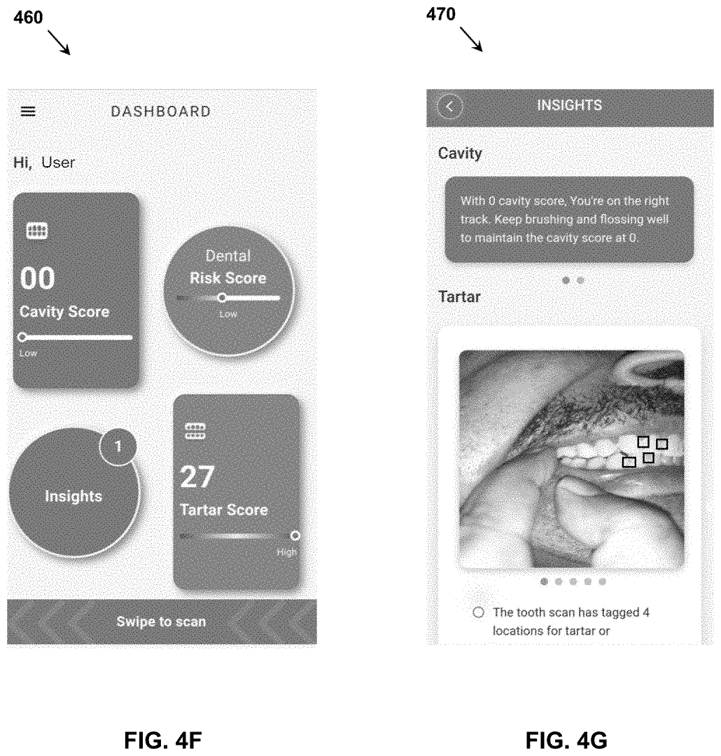

FIG. 2 illustrates a method (200) for self-assessment of dental problems, in accordance with an embodiment of the present invention. The method (200) would be better understood with reference to the information flow diagrams (300, 350) and screenshots (410-470) of the exemplary implementation shown in FIGS. 3A-3B and 4A-4G, respectively. For the purpose of explanation, the following exemplary implementation assumes that the user (302) is using a smartphone (304) having camera (104) and the user interface (106), for self-assessment. The processing may be done on the smartphone (304) itself or using the remote analytics server. Before starting the method (200) of operation, the user (302) is required to register with the system (100), using his/her personal details selected from one or more of, a name, age, email, phone number etc. The user (302) may be provided with or enabled to create unique ID and/or password (may be an OTP) for registration, to login to the system (100). After logging in, the user (302) is enabled to start the process of dental assessment. It will be understood to a skilled addressee that the steps of the method (200) may not be required to follow a strict order and may some of the steps actually be performed in a different order or even simultaneously.

The method (200) starts at step 202 by receiving a plurality of images and/or videos of the teeth of the user (302) from multiple angles captured from a camera (104). The same has been illustrated in FIGS. 3A and 4A. The multiple angles include such as, but not limited to, front teeth, upper jaw, lower jaw, left side and right side. A demo picture such as ones shown in FIGS. 4A and 4B may be provided to the user (302) to let him/her know how an ideal image looks and help him/her click the correct image. The user (302) is required to upload all the five images and the images may be checked for correct lighting and visibility in the images, before proceeding to the next step.

Then, at step 204, the processing module (102) is adapted to receive one or more inputs related to existing health conditions and habits of the user (302) possibly affecting the teeth. Herein, the existing health conditions possibly affecting the teeth, may include, but not limited to, high blood pressure, heart disease, diabetes, long-term kidney disease, lung disease, obesity, osteoporosis, rheumatoid arthritis, HIV and anemia. Also, the habits of the user (302) possibly affecting the teeth are selected from one or more of, but not limited to, brushing habits, flossing habits, smoking, alcohol consumption, tobacco consumption, excessive coffee consumption, binge eating, inadequate hydration, grinding the teeth, misuse of tooth picks and nail biting.

To receive the inputs from the user (302) in the form of, but not limited to, question-answers, MCQs, or may simply ask the user (302) to add his/her habits or health condition in a dialogue box. In one example, refer to FIG. 4C, the processing module (102) may provide a questionnaire with options to select, on the user interface (106) of the smartphone (304) to get relevant inputs form the user (302) regarding his habits and dental conditions. This is advantageous as the user (302) may not know which of his/her habits or health condition might be affecting the teeth. Like in FIG. 4C, the user (302) is asked about his/her diabetes, family history of cavities/gum infections, toothbrushing habits, making habits etc. So, the user (302) selects the relevant options or answers the queries accordingly.

Referring to FIG. 3B, at step 206, the plurality of images and/or videos are processed to identify dental condition and possible problems, on the received the plurality of images and/or videos. The processing module (102) utilizes the trained machine learning models (trained using thousands of images and still continue to improve with new assessments being done) to identify dental condition without the need for a dentist. It also gives information about possible problems that may occur in future if dental condition doesn't improve). In a simpler alternate embodiment, the processing module (102) may simply compare the received plurality of images/videos with the stored images indicative of various dental conditions & symptoms in the data repository (108), to identify the dental condition and problems. As previously mentioned, the dental condition and problems include dental cavities, dental plaque & tartar, missing teeth, gingivitis, bleeding gums, gum recession, gaps in the teeth, crooked teeth, teeth positioning, chipped or worn-out teeth, teeth discoloration and jaw alignment.

In addition, if there are problems identified in the teeth, then a dental risk area is also marked on the images for reference of the user (302). The marking may be done on the relevant image from the received images and then displayed on the smartphone (304) screen. The marking of each dental risk area may be done using virtual shapes/pointers. These virtual shape or pointers may be a circle, square, rectangle of variable sizes. However, multiple markers may also be used for marking one risk area, if the area is larger than a predetermined size. The same has been shown in FIG. 4D. For example: These images, containing the desired objects (e.g., Cavity, Tartar in FIGS. 4D and 4E), are then labelled by markings (bounding box/rectangles) around the objects.

After that, at step 208, the processor module is configured to correlate the identified dental condition & problems with the existing medical condition and habits of the user (302). It is well understood in the art that various illness or medical condition can be related to different dental problems. For example:
High blood pressure raises the risk of developing gum disease, so if one has problem of high BP, he/she may be more likely to have red and bleeding gums. Medications for high blood pressure can also affect your gums and cause dry mouth, which can lead to tooth decay.
Diabetes can cause periodontitis, the severe form of gum disease. The gums may start to pull away from the teeth, which can make them loose or even fall out.
Rheumatoid arthritis (RA) is also linked to gum disease by inflammation. Many studies suggest that RA increases the chance of developing gum disease and having gum disease increases the risk of RA. Studies show the more teeth a person loses, the more severe RA is likely to be.

The same is true for habits. The habits of a person also affect the dental health. For example:
Smoking (cigarettes and other tobacco products (including vaping)) stains teeth can cause gum and periodontal disease, and can even result in premature tooth loss. It can reduce the production of saliva, increasing the risk of bad breath (caused by harmful bacteria and food decomposition) and tooth decay.
Nail-Biting—Biting your nail does not only cause cracking, chipping, or wearing down of teeth but also causes jaw dysfunction. When biting the nails, the jaws are positioned at an unnatural angle by pushing the lower jaw out of line. Over time, this leads to temporomandibular disorder (TMD), a dental health condition characterized by a painful jaw.
Drinking Too Much Coffee—Like cigarette and tobacco smoking, coffee can also stain the teeth. Adding more sugar and drinking several cups of coffee every day can turn the teeth dull and yellow in colour. Sugar can also increase the risk of tooth decay.

So, just as above mentioned examples, there are many medical conditions and lifestyle habits that affect the dental health. Correlation explains how one or more variables are related to each other. These variables can be input data features which have been used to forecast our target variable. The Correlation, statistical technique which determines how one variables moves/changes in relation with the other variable. It gives us the idea about the degree of the relationship of the two variables. So, the processing module (102) correlates the health condition and problems identified in the previous step 206, with the medical conditions and habits of the user (302), to determine the risk and understand the dental conditions in a better manner. The processing module (102) may utilize one or more correlation algorithms for the same.

Based on the above, the processing module (102) is configured to generate assessment details along with a risk assessment score. The assessment details include, but not limited to, details of the identified dental condition and problems; correlation with the existing medical condition and habits; possible future problems in teeth; corrective changes in lifestyle and habits to improve oral hygiene; and if required, advice to seek consult a dentist. Further, the risk assessment score is generated by assigning a separate risk score to each identified dental condition & problem, and then generating an overall dental risk score also taking into consideration the existing medical condition and habits. This enables the user (302) to identify high, medium and low risk conditions.

Then, at step 250, the processing module (102) is configured to provide the assessment details and the risk assessment score along with marked images and/or videos showing the dental risk areas on the user interface (106).

As can be seen from the FIGS. 4D and 4E, the assessment details have been provided for tartar and cavities, respectively, in two of the images provided by the user (302). The screenshot (440) shows four tooth surfaces affected by tartar deposits. The risk score (mentioned on top) is also high. Whereas the screenshot (450) found 0 cavities and showed a low risk score. FIG. 4F shows a dashboard of the system (100), where overall summary of the assessment is provided with different scores for individual dental problems identified and also overall risk scores. “Insights” tab provided in FIG. 4F allow the user (302) to see the details of the identified dental condition, problems and suggestions by the system (100) (if any). The same has been illustrated in FIG. 4G. Using the risk score appropriate actions can be suggested to improve and monitor oral hygiene.

In accordance with an embodiment of the present invention, the processing module (102) further allows the user (302) to track and monitor the improvement of the respective oral hygiene by keeping results of previous assessments. Since details of previous assessment are also saved in the system (100) for respective user (302), so when a user (302) re-takes the assessment next time, the processing module (102) may provide a comparative analysis of the present assessment results with the previous assessment results, so that the user (302) can monitor the progress/decay of his/her dental health.

In accordance with an embodiment of the present invention, the processing module (102) is also configured to utilize sensory inputs from one or more of trans illumination lights, ultrasound or Infrared sensors, accelerometers and gyro sensors available in the smartphones or dedicated external sensors connected with processing module (102). These may be useful for providing more in-depth dental health analysis.

Apart from the above, following are some additional features of the present invention:

### Exposed Roots/Abrasion Defect Detection

It's a critical indicator for gum loss, poor brushing technique, and grinding and over clenching of jaws as a result of stress. Using this data, the system may recommend a right toothbrush for the patient.

### Virtual Smile Design

Using the smile pictures provided by the patient, the algorithm will do a instant virtual smile design and alignment visualization. This can be useful to make a decision on the type of braces as well as aligners.

### Tooth Color Determination

The algorithm can scan through the teeth images and will be able to identify the color of the teeth using the dental shade matching. This will be useful to determine the tooth shade identification for shade matching for whitening purposes or for prosthetic purposes.

The present invention finds a number of useful applications, some of which have been explained below:

The present invention can be extremely useful for dental insurers, as they often require prior check-up of the teeth before issuing an insurance policy etc. However, it is not feasible a person to go for medical check-up just to complete the formality for buying an insurance (especially in situations like the Covid pandemic etc.). So, in that scenario the present invention can enable a person to self-assess his/her dental health and submit dental assessment report to such insurance companies, to complete the formalities. This could also be used by expert dentists to identify pre-existing problems before the insurance. This in turn can save the insurance companies' huge amount of money because of the insurance claims.

Apart from the above, the present invention also provides a cost-effective solution for the dental professionals (individuals) that can be used access dental patients as a pre-check-up, remotely before or without physically checking the patients. The dental professionals can themselves identify by identifying early signs of problems or the extent of problems by referring to the images and marked dental risk areas.

Similarly, there may be many more possible applications of the present invention, without departing from the scope of the present invention.

However, it should be noted that the present invention should not be confused with diagnosis or treatment of dental problems. The present invention is a self-assessment tool that can help in reducing dental problems by identifying early signs of problems, manage as well as monitor dental cavities and also enable people to the assessment results with dental experts, if required, for further diagnosis.

It should also be understood that, unless specifically stated otherwise as apparent from the following discussion, it is appreciated that throughout the description, discussions utilizing terms such as “controlling” or “obtaining” or “computing” or “storing” or “receiving” or “determining” or the like, refer to the action and processes of a computer system, or similar electronic computing device, that processes and transforms data represented as physical (electronic) quantities within the computer system's registers and memories into other data similarly represented as physical quantities within the computer system memories or registers or other such information storage, transmission or display devices.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to be providing broadest scope of consistent with the principles and the novel and inventive features disclosed or suggested herein. Accordingly, the invention is anticipated to hold on to all other such alternatives, modifications, and variations that fall within the scope of the present invention and appended claims.

We claim:

1. A system for self-assessment of dental problems, the system comprising:
a camera adapted to capture a plurality of images and/or videos of teeth of a user from multiple angles;
a user interface to receive one or more inputs related to a medical condition and habits of the user affecting the teeth; and
a processing module connected with the camera and the user interface;
wherein the processing module is configured to:
receive the plurality of images and/or videos of the teeth of the user from multiple angles;
receive one or more inputs related to existing health conditions and habits of the user possibly affecting the teeth;
process the plurality of images and/or videos to identify a dental condition and possible problems and marking dental risk area, if any, on the received the plurality of images and/or videos;
correlate the identified dental condition and problems with the existing medical condition and habits of the user and accordingly generate assessment details along with a risk assessment score; and
provide the assessment details and the risk assessment score along with marked images and/or videos showing the dental risk areas on the user interface.

2. The system as claimed in claim 1, wherein the existing health conditions possibly affecting the teeth, include high blood pressure, heart disease, diabetes, long-term kidney disease, lung disease, obesity, osteoporosis, rheumatoid arthritis, HIV and anemia.

3. The system as claimed in claim 1, wherein the habits of the user possibly affecting the teeth are selected from one or more of brushing habits, flossing habits, smoking, alcohol consumption, tobacco consumption, excessive coffee consumption, binge eating, inadequate hydration, grinding the teeth, misuse of tooth picks and nail biting.

4. The system as claimed in claim 1, wherein the processing module is adapted to identify the dental condition and possible problems in permanent teeth, deciduous teeth, teeth with braces or any form of aligners and teeth with dental implants including all fixed and removable prosthesis.

5. The system as claimed in claim 1, wherein the dental condition and problems include dental cavities, dental plaque and tartar, missing teeth, gingivitis, bleeding gums, gum recession, gaps in the teeth, crooked teeth, teeth positioning, chipped or worn-out teeth, teeth discoloration and jaw alignment.

6. The system as claimed in claim 1, wherein the assessment details include details of the identified dental condition and problems; correlation with the existing medical condition and habits; possible future problems in teeth; corrective changes in lifestyle and habits to improve oral hygiene; and if required, advice to seek consult a dentist.

7. The system as claimed in claim 1, wherein the risk assessment score is generated by assigning a separate risk score to each identified dental condition and problem, and then generating an overall dental risk score also taking into consideration the existing medical condition and habits, thereby enabling the user to identify high, medium and low risk conditions.

8. The system as claimed in claim 1, wherein the processing module is configured to enable the user to track and monitor the improvement of the respective oral hygiene by keeping results of previous assessments and providing a comparative analysis of the present assessment results with the previous assessment results.

9. The system as claimed in claim 1, wherein the camera is selected from camera provided in a mobile computing device such as a smartphone, tablet or laptop; and a dedicated camera device such as DSLR, digital camera, mirrorless camera and webcam;
wherein, the processing module is also configured to utilize sensory inputs from one or more of transillumination lights, ultrasound or InfraRed sensors, accelerometers and gyro sensors available in the smartphones or dedicated external sensors connected with processing module.

10. A method for self-assessment of dental problems, the method comprising:
receiving a plurality of images and/or videos of the teeth of the user from multiple angles captured from a camera;
receiving, from a user interface, one or more inputs related to existing health conditions and habits of the user possibly affecting the teeth;
processing the plurality of images and/or videos to identify dental condition and possible problems and marking dental risk area, if any, on the received the plurality of images and/or videos;
correlating the identified dental condition and problems with the existing medical condition and habits of the user and accordingly generating assessment details along with a risk assessment score; and
providing the assessment details and the risk assessment score along with marked images and/or videos showing the dental risk areas on the user interface.
